(19) United States
(12) Patent Application Publication
(10) Pub. No.: US 2016/0022649 A1
(43) Pub. Date: Jan. 28, 2016

(54) USE OF INHIBITORS OF MTOR TO IMPROVE VASCULAR FUNCTIONS IN APOE4 CARRIERS

(71) Applicant: THE BOARD OF REGENTS OF THE UNIVERSITY OF TEXAS SYSTEM, Austin, TX (US)

(72) Inventors: Ai-Ling LIN, San Antonio, TX (US); Arlan RICHARDSON, San Antonio, TX (US); Veronica GALVAN, San Antonio, TX (US); Peter FOX, San Antonio, TX (US); Steven AUSTAD, San Antonio, TX (US); Kathleen FISCHER, San Antonio, TX (US)

(21) Appl. No.: 14/775,364

(22) PCT Filed: Mar. 14, 2014
(86) PCT No.: PCT/US14/28711

§ 371 Date: Sep. 11, 2015

Related U.S. Application Data
(60) Provisional application No. 61/790,485, filed on Mar. 15, 2013.

(51) Int. Cl. A61K 31/436; A61K 9/51; A23L 1/30; A61K 45/06

(52) U.S. Cl. CPC A61K 31/436; A61K 45/06; A61K 9/5146; A61K 9/5138; A23L 1/30; A23V 2002/00

(57) ABSTRACT

Disclosed are methods and compositions for preventing cerebrovascular function dysfunction in a patient who has been identified as an ApoE4 carrier. The disclosed methods and compositions include rapamycin, a rapamycin analog, or another such inhibitor of the target of rapamycin (TOR).

### CROSS-REFERENCE TO RELATED APPLICATIONS

This application claims the benefit of priority of U.S. Provisional Application No. 61/790,485, filed on Mar. 15, 2013, which is hereby incorporated by reference in its entirety

### GOVERNMENTAL RIGHTS

This invention was made with government support under agreement number AG036613 awarded by the National Institutes of Health (NIH). The government has certain rights in the invention.

### BACKGROUND OF THE INVENTION

A. Field of the Invention

The invention relates to methods and compositions for preventing cerebrovascular function dysfunction in a patient who has been identified as an ApoE4 carrier. The methods and compositions include rapamycin, rapamycin analogs, or other inhibitors of the mammalian target of rapamycin (“mTOR” or “mTORC1”).

B. Description of Related Art

Apolipoprotein E (ApoE) is a class of apolipoprotein found in the chylomicron and Intermediate-density lipoprotein (IDLs) that is essential for the normal catabolism of triglyceride-rich lipoprotein constituents. ApoE is polymorphic with three major isoforms: ApoE2 (cys112, cys158), ApoE3 (cys112, arg158), and ApoE4 (arg112, arg158).

ApoE4 is found in approximately 14 percent of the population, and this polymorph has been implicated in atherosclerosis, Alzheimer's disease, impaired cognitive function, reduced hippocampal volume, faster disease progression in Multiple Sclerosis, unfavorable outcome after traumatic brain injury, ischemic cerebrovascular disease, sleep apnea, and reduced neurite outgrowth. Although ApoE4 is known as a prevalent genetic risk factor for Alzheimer's disease (AD), stroke, depression, and poor prognosis from TBI, there is currently no therapy available to reduce the risk to these outcomes for carriers of ApoE4.

### SUMMARY OF THE INVENTION

In some aspects, provided are methods for preventing cerebrovascular dysfunction in a patient comprising administering an effective amount of a composition comprising rapamycin or an analog thereof to a patient who has been identified as an ApoE4 carrier. In some embodiments, preventing the cerebrovascular function dysfunction prevents Alzheimer's Disease (AD), non-AD dementia, age-related cognitive dysfunction, stroke, depression, or cerebral palsy. In some embodiments, provided are methods for treating traumatic brain injury (TBI) in a patient comprising administering an effective amount of a composition comprising rapamycin or an analog thereof to a patient who has been identified as an ApoE4 carrier.

In some embodiments, the rapamycin or analog thereof are encapsulated or coated, or the composition comprising the rapamycin or analog thereof is encapsulated or coated. In some embodiments, the encapsulant or coating may be an enteric coating. In some embodiments, the encapsulant or coating may be an enteric coating. In some embodiments, the coating comprises cellulose acetate succinate, hydroxy propyl methyl cellulose phthalate co-polymer, or a polymethacrylate-based copolymer selected from the group consisting of methyl acrylate-methacrylic acid copolymer, and a methyl methacrylate-methacrylic acid copolymer. In some embodiments, the coating comprises Poly(methacylic acid-co-ethyl acrylate) in a 1:1 ratio, Poly(methacrylic acid-co-ethyl acrylate) in a 1:1 ratio, Poly(methacylic acid-co-methyl methacrylate) in a 1:1 ratio, Poly(methacylic acid-co-methyl methacrylate) in a 1:2 ratio, Poly(methyl acrylate-co-methyl methacrylate-co-methacrylic acid) in a 7:3:1 ratio, Poly(ethyl acrylate-co-methyl methacrylate-co-trimethylammonioethyl methacrylate chloride) in a 1:2:0.2 ratio, Poly(ethyl acrylate-co-methyl methacrylate-co-trimethylammonioethyl methacrylate chloride) in a 1:2:0.1 ratio, or Poly(butyl methacylate-co-(2-dimethylaminoethyl) methacrylate-co-methyl methacrylate) in a 1:2:1 ratio, a naturally-derived polymer, or a synthetic polymer, or any combination thereof. In some embodiments, the naturally-derived polymer is selected from the group consisting of alginates and their various derivatives, chitosans and their various derivatives, carrageenans and their various analogues, celluloses, gums, gelatins, pectins, and gellans. In some embodiments, the naturally-derived polymer is selected from the group consisting of polyethyleneglycols (PEGs) and polyethyleneoxides (PEOs), acrylic acid homo- and copolymers with acrylates and methacrylates, homopolymers of acrylates and methacrylates, polyvinyl alcohol PVOH), and polyvinyl pyrrolidone (PVP).

An effective amount of rapamycin or rapamycin analog or derivative will depend upon the disease to be treated, the length of duration desired and the bioavailability profile of the implant, and the site of administration. In some embodiments, the composition comprises rapamycin or an analog thereof at a concentration of 0.001 mg to 30 mg total per dose. In some embodiments, the composition comprising rapamycin or an analog of rapamycin comprises 0.001% to 60% by weight of rapamycin or an analog of rapamycin. In some embodiments, the average blood level of rapamycin in the subject is greater than 0.5 ng per ml, whole blood after administration of the composition.

The composition can be administered to the subject using any method known to those of ordinary skill in the art. In some embodiments, the composition may be administered intravenously, intracerebrally, intracranially, intraventricularly, intrathecally, into the cortex, thalamus, hypothalamus, hippocampus, basal ganglia, substantia nigra or the region of the substantia nigra, cerebellum, intradermally, intraarterially, intraperitoneally, intralesionally, intratracheally, intranasally, topically, intramuscularly, intraperitoneally, anally, subcutaneously, orally, topically, locally, inhalation (e.g., aerosol inhalation), injection, infusion, continuous infusion, localized perfusion bathing target cells directly, via a catheter, via a lavage, in creams, in lipid compositions (e.g., liposomes), or by other method or any combination of the forgoing as would be known to one of ordinary skill in the art. In some embodiments, the composition is administered orally, intravenously, enterically, or intranasally. In some embodiments, the composition comprising rapamycin or an analog of rapamycin is comprised in a food or food additive.

The dose can be repeated as needed as determined by those of ordinary skill in the art. In some embodiments, the rapamycin or analog of rapamycin is administered in two or more doses. Where more than one dose is administered to a subject, the time interval between doses can be any time interval as determined by those of ordinary skill in the art. For example, the two doses may be 1, 2, 3, 4, 5, 6, 7, 8, 9, 10, 11, 12, 13, 14, 15, 16, 17, 18, 29, 20, 21, 22, 23, or 24 hours apart, or any range therein. In some embodiments, the composition may be administered daily, weekly, monthly, annually, or any range therein. In some embodiments, the interval of time between administration of doses comprising rapamycin or an analog of rapamycin is between 0.5 to 30 days.

In some embodiments, the method comprises further administering one or more secondary or additional forms of therapies. In some embodiments, the subject is further administered a composition comprising a second active agent. In some embodiments, the second active agent is endothelial nitric oxide synthase (eNOS), a cholinesterase inhibitor, an anti-glutamate, an anti-hypertensive agent, an anti-platelet agent, an antihyperlipidemic agent, an anti-anxiety agent, an anti-depressant agent, an antipsychotic agent, an anti-seizure agent, an anti-Parkinson agent, an anti-spasmodic agent, an anti-tremor agent, a muscle relaxant agent, or a medication that alleviates or treats low blood pressure, cardiac arrhythmia, or diabetes; or a biological agent that includes an antibody or antibodies to neurofibrillary tangles or cerebral plaques. In some embodiments, the anti-cholinesterase therapeutic is tacrine, donepezil, rivastigmine, galantamine, or a humanized antibody, protein, or RNA sequence. In some embodiments, the anti-glutamate therapeutic is memantine, or a humanized antibody, protein or RNA sequence. In some embodiments, the biologic agent to neurofibrillary tangles or cerebral plaques is a polyclonal antibody or humanized monoclonal antibody, protein or RNA sequence.

In some embodiments, the composition comprising rapamycin or an analog of rapamycin is administered at the same time as the composition comprising the second active agent. In some embodiments, the composition comprising rapamycin or an analog of rapamycin is administered before or after the composition comprising the second active agent is administered. In some embodiments, the two treatments may be 1, 2, 3, 4, 5, 6, 7, 8, 9, 10, 11, 12, 13, 14, 15, 16, 17, 18, 29, 20, 21, 22, 23, or 24 hours apart, 1, 2, 3, 4, 5, 6, 7, 8, 9, 10, 11, 12, 13, 14, 15, 16, 17, 18, 29, 20, 21, 22, 23, 24, 25, 26, 27, 28, 29, 30, or 31 days apart, 1, 2, 3, 4, 5, 6, 7, 8, 9, 10, 11, or 12 months apart, or one or more years apart or any range therein. In some embodiments, the interval of time between administration of composition comprising rapamycin or an analog of rapamycin and the composition comprising the second active agent is 1 to 30 days.

In some embodiments, the mTOR inhibitor or an analog thereof is eRapa. “eRapa” is generically used to refer to encapsulated or coated forms of Rapamycin or other mTOR inhibitors or their respective analogs disclosed herein and equivalents thereof. In some embodiments, the encapsulant or coating used for and incorporated in eRapa preparation may be an enteric coating. In some embodiments, the mTOR inhibitor or analog thereof is nanoRapa. “nanoRapa” is generically used to refer to the rapamycins, rapamycin analogs, or related compositions within the eRapa preparation are provided in the form of nanoparticles that include the rapamycin or other mTOR inhibitor. In some embodiments, the mTOR inhibitor or analog thereof is e-nanoRapa. “e-nanoRapa” is generically used to refer to eRapa variations formed from nanoRapa particles. After preparing the nanoRapa preparations, the nanoRapa preparation may then be coated with an enteric coating, to provide an eRapa preparation formed from nanoRapa particles.

In some embodiments, the eRapa, nanoRapa, or e-nanoRapa is encased in a coating comprising cellulose acetate succinate, hydroxy propyl methyl cellulose phthalate co-polymer, or a polymethacrylate-based copolymer selected from the group consisting of methyl acrylate-methacrylic acid copolymer, and a methyl methacrylate-methacrylic acid copolymer. In some embodiments, the coating comprises Poly(methacylic acid-co-ethyl acrylate) in a 1:1 ratio, Poly(methacrylic acid-co-ethyl acrylate) in a 1:1 ratio, Poly(methacylic acid-co-methyl methacrylate) in a 1:1 ratio, Poly(methacylic acid-co-methyl methacrylate) in a 1:2 ratio, Poly(methyl acrylate-co-methyl methacrylate-co-methacrylic acid) in a 7:3:1 ratio, Poly(ethyl acrylate-co-methyl methacrylate-co-trimethylammonioethyl methacrylate chloride) in a 1:2:0.2 ratio, Poly(ethyl acrylate-co-methyl methacrylate-co-trimethylammonioethyl methacrylate chloride) in a 1:2:0.1 ratio, or Poly(butyl methacylate-co-(2-dimethylaminoethyl) methacrylate-co-methyl methacrylate) in a 1:2:1 ratio, a naturally-derived polymer, or a synthetic polymer, or any combination thereof. In some embodiments, the naturally-derived polymer is selected from the group consisting of alginates and their various derivatives, chitosans and their various derivatives, carrageenans and their various analogues, celluloses, gums, gelatins, pectins, and gellans. In some embodiments, the naturally-derived polymer is selected from the group consisting of polyethyleneglycols (PEGs) and polyethyleneoxides (PEOs), acrylic acid homo- and copolymers with acrylates and methacrylates, homopolymers of acrylates and methacrylates, polyvinyl alcohol PVOH), and polyvinyl pyrrolidone (PVP).

In some embodiments, the composition comprises eRapa or an analog thereof at a concentration of at or between 50 micrograms and 200 micrograms per kilogram for daily administration, or the equivalent for other frequencies of administration.

In some embodiments, the eRapa, nanoRapa, or e-nanoRapa is administered orally, enterically, colonically, anally, intravenously, or dermally with a patch. In some embodiments, the eRapa, nanoRapa, or e-nanoRapa is administered in two or more doses. In some embodiments, the interval of time between administration of doses comprising eRapa, nanoRapa, or e-nanoRapa is 0.5 to 30 days. In some embodiments, the interval of time between administration of doses comprising eRapa, nanoRapa, or e-nanoRapa is 0.5 to 1 day. In some embodiments, the interval of time between administration of doses comprising eRapa, nanoRapa, or e-nanoRapa is 1 to 3 days. In some embodiments, the interval of time between administration of doses comprising eRapa, nanoRapa, or e-nanoRapa is 1 to 5 days. In some embodiments, the interval of time between administration of doses comprising eRapa, nanoRapa, or e-nanoRapa is 1 to 7 days. In some embodiments, the interval of time between administration of doses comprising eRapa, nanoRapa, or e-nanoRapa is 1 to 15 days.

In some embodiments, the subject is further administered a composition comprising a second active agent. In some embodiments, the second active agent is metformin, celocoxib, eflornithine, sulindac, ursodeoxycholic acid, an anti-inflammatory agent, an anti-autoimmune agent, or a cytotoxic or cytostatic anti-cancer agent. In some embodiments, the composition comprising eRapa, nanoRapa, or e-nanoRapa is administered at the same time as the composition comprising the second active agent. In some embodiments, the composition comprising eRapa, nanoRapa, or e-nanoRapa is administered before or after the composition comprising the second active agent is administered. In some embodiments, the interval of time between administration of composition comprising eRapa, nanoRapa, or e-nanoRapa and the composition comprising the second active agent is 1 to 30 days.

In some embodiments, the composition comprising eRapa, nanoRapa, or e-nanoRapa prevents cerebrovascular dysfunction in a patient who has been identified as an ApoE4 carrier. In some embodiments, preventing the cerebrovascular function dysfunction prevents Alzheimer's Disease (AD), non-AD dementia, age-related cognitive dysfunction, stroke, depression, or cerebral palsy. In some embodiments, provided are methods for treating traumatic brain injury (TBI) in a patient comprising administering an effective amount of a composition comprising rapamycin or an analog thereof to a patient who has been identified as an ApoE4 carrier.

In some embodiments, the composition comprising eRapa, nanoRapa, or e-nanoRapa is comprised in a food or food additive.

Unless otherwise specified, the percent values expressed herein are weight by weight and are in relation to the total composition.

The term “about” or “approximately” are defined as being close to as understood by one of ordinary skill in the art, and in one non-limiting embodiment the terms are defined to be within 10%, preferably within 5%, more preferably within 1%, and most preferably within 0.5%.

The terms “inhibiting,” “reducing,” “treating,” or any variation of these terms, includes any measurable decrease or complete inhibition to achieve a desired result. Similarly, the term “effective” means adequate to accomplish a desired, expected, or intended result.

The terms “prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

The use of the word “a” or “an” when used in conjunction with the term “comprising” may mean “one,” but it is also consistent with the meaning of “one or more,” “at least one,” and “one or more than one.”

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. in relation to the total composition.

The compositions and methods for their use can “comprise,” “consist essentially of,” or “consist of” any of the ingredients or steps disclosed throughout the specification. With respect to the transitional phrase “consisting essentially of,” in one non-limiting aspect, a basic and novel characteristic of the compositions and methods is the ability of eRapa, e-nanoRapa or other rapamycin preparations to prevent cerebrovascular function dysfunction in a patient who has been identified as an ApoE4 carrier.

It is contemplated that any embodiment discussed in this specification can be implemented with respect to any method or composition of the invention, and vice versa. Furthermore, compositions of the invention can be used to achieve methods of the invention.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1E. Rapamycin effects on cerebral blood flow (CBF) in LDL knockout and Alzheimer-s disease (AD)-like mice. Rapamycin significantly enhanced CBF in (A) high-fat diet LDL knockout mice and (B) AD-like transgenic mice. (C) Mouse brain atlas showing the hippocampus. (D) Quantitative global and hippocampal CBF in the LDL knockout mice. (E) Quantitative global and hippocampal CBF in the AD-like transgenic mice.

FIG. 2. Rapamycin effects on cerebral blood flow (CBF) in healthy aging rats. (Top) The CBF heatmap of adult control rats (14 months of age), old control rats (32 months of age) and old rapa-fed rats (32 months of age, 14 ppm rapamycin). (Bottom) The quantitative CBF in the whole brain, cortex and hippocampus of the rats. CBF was reduced significantly with age, and rapamycin restored CBF in old rats.

FIG. 3 depicts an embodiment of methods of the present invention, showing a sequence of steps for producing nanoRapa rapamycin nanoparticles by stirring a mixture of a combination of rapamycin and a water-miscible solvent with a combination of water and dispersants.

FIG. 4 depicts an embodiment of methods of the present invention, showing a sequence of steps for producing e-nanoRapa microencapsulated nanoparticles of rapamycin.

FIG. 5 depicts a nanoRapa embodiment illustrating a detailed view of a micelle created by particular dispersants in solution as is used as part of a sequence of fabricating the nanoRapa rapamycin nanoparticles.

FIG. 6 depicts particular e-nanoRapa embodiments of the invention, particularly with reference to fabrication of e-nanoRapa microencapsulated nanoparticles of rapamycin as produced by the method of FIG. 4.

### DESCRIPTION OF ILLUSTRATIVE EMBODIMENTS

The inventors have discovered an effective therapy for preventing cerebrovascular dysfunction in a patient comprising administering an effective amount of a composition comprising rapamycin or an analog thereof to a patient who has been identified as an ApoE4 carrier comprising administration of rapamycin, an analog of rapamycin, or another inhibitor of mTOR. In certain embodiments, the rapamycin, an analog of rapamycin, or other inhibitor of mTOR is administered orally. In certain embodiments, the rapamycin, an analog of rapamycin, or other inhibitor of mTOR is administered in the form of an eRapa and/or e-nanoRapa preparation.

The apolipoprotein ε4 (ApoE4) allele is a major a risk factor for Alzheimer's Disease (AD) as well as neurovascular and psychiatric disorders associated with alterations in cerebrovascular function, e.g., non-AD dementia and age-related cognitive dysfunction, stroke, depression, cerebral palsy, and recovery from traumatic brain injury (TBI) with and without hemorrhage. For example, individuals who possess one or two ApoE4 alleles have a 3- to 8-fold higher possibility of developing AD compared to non-carriers. Recent studies have shown that an early phenotype (e.g., shown at 20-30 years of age) of ApoE4 carriers is cerebrovascular dysfunction as assessed by cerebral blood flow (CBF) measurements.

Cerebrovascular dysfunction has been proposed to be an initiating event leading to alteration in neuronal activity (related to psychiatric disorders), production of proinflammatory cytokines, and eventually 0 amyloid deposition and loss of memory (related to AD). Cerebrovascular dysfunction could also play a role in the higher risk to stroke and TBI observed in ApoE4 carriers.

Vascular pathology causes or contributes to dementia in a substantial portion of patients. Cerebral microbleeds are present in patients with vascular dementia and with Alzheimer's disease (AD) (Iadecola 2004). Cerebral microbleeds and macroscopic hemorrhage are frequently consequences of cerebral amyloid angiopathy (CAA), which arises from the deposition of amyloid-β peptide (AB) in blood vessels (Fryer 2003). The vast majority of AD patients show CAA. CAA is also associated with Parkinson's disease and with dementia with Lewy bodies, and is strongly linked to cognitive decline in these disorders. The inventors have found that chronic inhibition of TOR by rapamycin treatment after disease onset negated brainvascular breakdown through activation of endothelial nitric oxide synthase (eNOS) in vascular endothelium, reduced cerebral amyloid angiopathy and microhemorrhages, decreased amyloid burden, and improved cognitive function in symptomatic AD mice.

The inventors have shown that rapamycin can restore vascular function and increase CBF in several rodent models that are associated with a decline in memory and increased depression, which supports the finding that rapamycin and other mTOR inhibitors will prevent/restore cerebrovascular dysfunction in ApoE4 carriers, thereby reducing the risk of AD as well as other neurovascular and psychiatric disorders associated with alterations in ApoE4 carriers, such as non-AD dementia and age-related cognitive dysfunction, stroke, depression, and cerebral palsy. In addition, rapamycin and other mTOR inhibitors can be used as a therapy in ApoE4 carriers exposed to traumatic brain injury (TBI) with and without hemorrhage brain trauma.

### A. ApoE4

Apolipoprotein E (ApoE) is a class of apolipoprotein found in the chylomicron and Intermediate-density lipoprotein (IDLs) that is essential for the normal catabolism of triglyceride-rich lipoprotein constituents. APOE is 299 amino acids long and transports lipoproteins, fat-soluble vitamins, and cholesterol into the lymph system and then into the blood. In peripheral tissues, ApoE is primarily produced by the liver and macrophages, and mediates cholesterol metabolism in an isoform-dependent manner. In the central nervous system, ApoE is mainly produced by astrocytes, and transports cholesterol to neurons via ApoE receptors, which are members of the low density lipoprotein receptor gene family.

The protein, ApoE, is mapped to chromosome 19 in a cluster with Apolipoprotein C1 and the Apolipoprotein C2. The APOE gene consists of four exons and three introns, totaling 3597 base pairs. ApoE is transcriptionally activated by the liver X receptor (an important regulator of cholesterol, fatty acid, and glucose homeostasis) and peroxisome proliferator-activated receptory, nuclear receptors that form heterodimers with Retinoid X receptors. In melanocytic cells APOE gene expression may be regulated by MITF. ApoE is polymorphic with three major isoforms: ApoE2 (cys112, cys158), ApoE3 (cys112, arg158), and ApoE4 (arg112, arg158). Although these allelic forms differ from each other by only one or two amino acids at positions 112 and 158, these differences alter apoE structure and function.

ApoE4 is found in approximately 14 percent of the population, and has been implicated in atherosclerosis, Alzheimer's disease, impaired cognitive function, reduced hippocampal volume, faster disease progression in Multiple Sclerosis, unfavorable outcome after traumatic brain injury, ischemic cerebrovascular disease, sleep apnea, and reduced neurite outgrowth. Although ApoE4 is known as a prevalent genetic risk factor for Alzheimer's disease (AD), stroke, depression and poor prognosis from TBI, there is currently no therapy available to reduce the risk to these outcomes for carriers of ApoE4.

In some aspects, methods of preventing cerebrovascular function dysfunction in a patient who has been identified as an ApoE4 carrier are provided. These methods may include administration of an effective amount of a composition comprising an mTOR inhibitor such as rapamycin or an analog thereof.

### B. Cognitive Impairment

Dementia or cognitive impairment refers to a set of symptoms that occur due to an underlying condition or disorder that causes loss of brain function. Dementia or cognitive impairment symptoms include difficulty with language, memory, perception, emotional behavior, personality (including changes in personality), or cognitive skills (including calculation, abstract thinking, problem-solving, and judgment). Dementia or cognitive impairment may be caused by a variety of underlying disorders, including Alzheimer's disease, Parkinson's disease, vascular pathology (which causes vascular cognitive impairment), Lewy Body disease (which causes Lewy Body dementia), and Pick's disease (which causes Frontotemporal dementia).

The major causes of dementia or cognitive impairment are Alzheimer's disease, Lewy Body disease, and vascular pathology. Vascular pathology is believed to account for 20-30% of dementia cases, and because vascular cognitive impairment is likely underdiagnosed, it may be even more common than previously thought. A common cause of vascular cognitive impairment is the occurrence of multiple small strokes (called “mini-strokes”) that affect blood vessels and nerve fibers in the brain, which ultimately promotes symptoms of dementia or vascular cognitive impairment. Thus, vascular cognitive impairment is more common in those patients who are at risk for stroke, such as elderly patients, or patients having high blood pressure, high cholesterol, high blood sugar, or an autoimmune or inflammatory disease (such as lupus or temporal arteritis).

Vascular cognitive impairment is a cognitive impairment that results from underlying vascular pathology. The term “vascular cognitive impairment” refers to various defects caused by an underlying vascular pathology, disease, disorder, or condition that affects the brain. For example, strokes, conditions that damage or block blood vessels, or disorders such as hypertension or small vessel disease may cause vascular cognitive impairment. As used herein, the term “vascular cognitive impairment” includes mild defects, such as the milder cognitive symptoms that may occur in the earliest stages in the development of dementia, as well as the more severe cognitive symptoms that characterize later stages in the development of dementia.

The various defects that may manifest as vascular cognitive impairment include mental and emotional symptoms (slowed thinking, memory problems, general forgetfulness, unusual mood changes such as depression or irritability, hallucinations, delusions, confusion, personality changes, loss of social skills, and other cognitive defects); physical symptoms (dizziness, leg or arm weakness, tremors, moving with rapid/shuffling steps, balance problems, loss of bladder or bowel control); or behavioral symptoms (slurred speech, language problems such as difficulty finding the right words for things, getting lost in familiar surroundings, laughing or crying inappropriately, difficulty planning, organizing, or following instructions, difficulty doing things that used to come easily, reduced ability to function in daily life).

### C. Traumatic Brain Injury

Traumatic brain injury (TBI), also known as intracranial injury, occurs when an external force traumatically injures the brain. TBI can be classified based on severity, mechanism (closed or penetrating head injury), or other features (e.g., occurring in a specific location or over a widespread area). Head injury usually refers to TBI, but is a broader category because it can involve damage to structures other than the brain, such as the scalp and skull.

TBI can cause a host of physical, cognitive, social, emotional, and behavioral effects, and outcome can range from complete recovery to permanent disability or death. Depending on the injury, treatment required may be minimal or may include interventions such as medications, emergency surgery or surgery years later. TBI can occur with and without hemorrhage brain trauma.

D. mTOR Inhibitors and Rapamycin

Any inhibitor of mTORC1 is contemplated for inclusion in the present compositions and methods. In particular embodiments, the inhibitor of mTORC1 is rapamycin or an analog of rapamycin. Rapamycin (also known as sirolimus and marketed under the trade name Rapamune) is a known macrolide. The molecular formula of rapamycin is C₅₁H₇₉NO₁₃. In some embodiments, the inhibitor of mTORC1 is rapamycin or an analog of rapamycin is administered orally in the form of an eRapa and/or e-nanoRapa preparation.

Rapamycin binds to a member of the FK binding protein (FKBP) family, FKBP 12. The rapamycin/FKBP 12 complex binds to the protein kinase mTOR to block the activity of signal transduction pathways. Because the mTOR signaling network includes multiple tumor suppressor genes, including PTEN, LKB1, TSC1, and TSC2, and multiple proto-oncogenes including PI3K, Akt, and eEF4E, mTOR signaling plays a central role in cell survival and proliferation. Binding of the rapamycin/FKBP complex to mTOR causes arrest of the cell cycle in the G1 phase (Janus et al., 2005).

mTORC1 inhibitors also include rapamycin analogs. Many rapamycin analogs are known in the art. Non-limiting examples of analogs of rapamycin include, but are not limited to, everolimus, tacrolimus, CCI-779, ABT-578, AP-23675, AP-23573, AP-23841, 7-epi-rapamycin, 7-thiomethyl-rapamycin, 7-epi-trimethoxyphenyl-rapamycin, 7-epi-thiomethyl-rapamycin, 7-demethoxy-rapamycin, 32-demethoxy-rapamycin, 2-desmethyl-rapamycin, and 42-O-(2-hydroxy)ethyl rapamycin.

Other analogs of rapamycin include: rapamycin oximes (U.S. Pat. No. 5,446,048); rapamycin aminoesters (U.S. Pat. No. 5,130,307); rapamycin dialdehydes (U.S. Pat. No. 6,680,330); rapamycin 29-enols (U.S. Pat. No. 6,677,357); O-alkylated rapamycin derivatives (U.S. Pat. No. 6,440,990); water soluble rapamycin esters (U.S. Pat. No. 5,955,457); alkylated rapamycin derivatives (U.S. Pat. No. 5,922,730); rapamycin amidino carbamates (U.S. Pat. No. 5,637,590); biotin esters of rapamycin (U.S. Pat. No. 5,504,091); carbamates of rapamycin (U.S. Pat. No. 5,567,709); rapamycin hydroxyesters (U.S. Pat. No. 5,362,718); rapamycin 42-sulfonates and 42-(N-carbalkoxy)sulfamates (U.S. Pat. No. 5,346,893); rapamycin oxepane isomers (U.S. Pat. No. 5,344,833); imidazolidyl rapamycin derivatives (U.S. Pat. No. 5,310,903); rapamycin alkoxyesters (U.S. Pat. No. 5,233,036); rapamycin pyrazoles (U.S. Pat. No. 5,164,399); acyl derivatives of rapamycin (U.S. Pat. No. 4,316,885); reduction products of rapamycin (U.S. Pat. Nos. 5,102,876 and 5,138,051); rapamycin amide esters (U.S. Pat. No. 5,118,677); rapamycin fluorinated esters (U.S. Pat. No. 5,100,883); rapamycin acetals (U.S. Pat. No. 5,151,413); oxorapamycins (U.S. Pat. No. 6,399,625); and rapamycin silyl ethers (U.S. Pat. No. 5,120,842).

Other analogs of rapamycin include those described in U.S. Pat. Nos. 6,015,809; 6,004,973; 5,985,890; 5,955,457; 5,922,730; 5,912,253; 5,780,462; 5,665,772; 5,637,590; 5,567,709; 5,563,145; 5,559,122; 5,559,120; 5,559,119; 5,559,112; 5,550,133; 5,541,192; 5,541,191; 5,532,355; 5,530,121; 5,530,007; 5,525,610; 5,521,194; 5,519,031; 5,516,780; 5,508,399; 5,508,290; 5,508,286; 5,508,285; 5,504,291; 5,504,204; 5,491,231; 5,489,680; 5,489,595; 5,488,054; 5,486,524; 5,486,523; 5,486,522; 5,484,791; 5,484,790; 5,480,989; 5,480,988; 5,463,048; 5,446,048; 5,434,260; 5,411,967; 5,391,730; 5,389,639; 5,385,910; 5,385,909; 5,385,908; 5,378,836; 5,378,696; 5,373,014; 5,362,718; 5,358,944; 5,346,893; 5,344,833; 5,302,584; 5,262,424; 5,262,423; 5,260,300; 5,260,299; 5,233,036; 5,221,740; 5,221,670; 5,202,332; 5,194,447; 5,177,203; 5,169,851; 5,164,399; 5,162,333; 5,151,413; 5,138,051; 5,130,307; 5,120,842; 5,120,727; 5,120,726; 5,120,725; 5,118,678; 5,118,677; 5,100,883; 5,023,264; 5,023,263; 5,023,262; all of which are incorporated herein by reference. Additional rapamycin analogs and derivatives can be found in the following U.S. Patent Application Pub. Nos., all of which are herein specifically incorporated by reference: 20080249123, 20080188511; 20080182867; 20080091008; 20080085880; 20080069797; 20070280992; 20070225313; 20070203172; 20070203171; 20070203170; 20070203169; 20070203168; 20070142423; 20060264453; and 20040010002.

Rapamycin or a rapamycin analog can be obtained from any source known to those of ordinary skill in the art. The source may be a commercial source, or natural source. Rapamycin or a rapamycin analog may be chemically synthesized using any technique known to those of ordinary skill in the art. Non-limiting examples of information concerning rapamycin synthesis can be found in Schwecke et al., 1995; Gregory et al., 2004; Gregory et al., 2006; Graziani, 2009.

### E. Encapsulated Rapamycin Compositions

In some aspects, the compositions comprising an inhibitor of mTOR are encapsulated or coated to provide eRapa preparations. In some embodiments, the encapsulant or coating may be an enteric coating. In some embodiments, the compositions comprising an inhibitor of mTOR are provided in the form of nanoRapa nanoparticles, and such nanoRapa nanoparticles are encapsulated or coated to provide e-nanoRapa preparations, which are relatively stable and beneficial for oral administration.

Many pharmaceutical dosage forms irritate the stomach due to their chemical properties or are degraded by stomach acid through the action of enzymes, thus becoming less effective. The coating may be an enteric coating, a coating that prevents release and absorption of active ingredients until they reach the intestine. “Enteric” refers to the small intestine, and therefore enteric coatings facilitate delivery of agents to the small intestine. Some enteric coatings facilitate delivery of agents to the colon. In some embodiments, the enteric coating is a EUDRAGIT (®) coating. Eudragit coatings include Eudragit L100-55 (for delivery to the duodenum), Poly(methacylic acid-co-ethyl acrylate) 1:1; Eudragit L 30 D-55 (for delivery to the duodenum), Poly(methacrylic acid-co-ethyl acrylate) 1:1; Eudragit L 100 (for delivery to the jejunum), Poly(methacylic acid-co-methyl methacrylate) 1:1; Eudragit S100 (for delivery to the ileum), Poly(methacylic acid-co-methyl methacrylate) 1:2; Eudragit FS 30D (for colon delivery), Poly(methyl acrylate-co-methyl methacrylate-co-methacrylic acid) 7:3:1; Eudragit RL (for sustained release), Poly(ethyl acrylate-co-methyl methacrylate-co-trimethylammonioethyl methacrylate chloride) 1:2:0.2; Eudragit RS (for sustained release), Poly(ethyl acrylate-co-methyl methacrylate-co-trimethylammonioethyl methacrylate chloride) 1:2:0.1; and Eudragit E (for taste masking), Poly(butyl methacylate-co-(2-dimethylamino ethyl) methacrylate-co-methyl methacrylate) 1:2:1. Other coatings include ethylcellulose and polyvinyl acetate. Benefits include pH-dependent drug release, protection of active agents sensitive to gastric fluid, protection of gastric mucosa from active agents, increase in drug effectiveness, good storage stability, and GI and colon targeting, which minimizes risks associated with negative systemic effects.

Some examples of enteric coating components include cellulose acetate pthalate, methyl acrylate-methacrylic acid copolymers, cellulose acetate succinate, hydroxy propyl methyl cellulose phthalate, hydroxy propyl methyl cellulose acetate succinate, polyvinyl acetate phthalate, methyl methacrylate-methacrylic acid copolymers, sodium alginate, and stearic acid. The coating may include suitable hydrophilic gelling polymers including but not limited to cellulosic polymers, such as methylcellulose, carboxymethylcellulose, hydroxypropylcellulose, hydroxypropylmethylcellulose, hydroxyethylcellulose, and the like; vinyl polymers, such as polyvinylpyrrolidone, polyvinyl alcohol, and the like; acrylic polymers and copolymers, such as acrylic acid polymer, methacrylic acid copolymers, ethyl acrylate-methyl methacrylate copolymers, natural and synthetic gums, such as guar gum, arabic gum, xanthan gum, gelatin, collagen, proteins, polysaccharides, such as pectin, pectic acid, alginic acid, sodium alginate, polyaminoacids, polyalcohols, polyglycols; and the like; and mixtures thereof. Any other coating agent known to those of ordinary skill in the art is contemplated for inclusion in the coatings of the microcapsules set forth herein.

The coating may optionally comprises a plastisizer, such as dibutyl sebacate, polyethylene glycol and polypropylene glycol, dibutyl phthalate, diethyl phthalate, triethyl citrate, tributyl citrate, acetylated monoglyceride, acetyl tributyl citrate, triacetin, dimethyl phthalate, benzyl benzoate, butyl and/or glycol esters of fatty acids, refined mineral oils, oleic acid, castor oil, corn oil, camphor, glycerol and sorbitol or a combination thereof. The coating may optionally include a gum. Non-limiting examples of gums include homopolysaccharides such as locust bean gum, galactans, mannans, vegetable gums such as alginates, gum karaya, pectin, agar, tragacanth, accacia, carrageenan, tragacanth, chitosan, agar, alginic acid, other polysaccharide gums (e.g., hydrocolloids), acacia catechu, salai guggal, indian bodellum, copaiba gum, asafetida, cambi gum, Enterolobium cyclocarpum, mastic gum, benzoin gum, sandarac, gambier gum, butea frondosa (Flame of Forest Gum), myrrh, konjak mannan, guar gum, welan gum, gellan gum, tara gum, locust bean gum, carageenan gum, glucomannan, galactan gum, sodium alginate, tragacanth, chitosan, xanthan gum, deacetylated xanthan gum, pectin, sodium polypectate, gluten, karaya gum, tamarind gum, ghatti gum, Accaroid/Yacca/Red gum, dammar gum, juniper gum, ester gum, ipil-ipil seed gum, gum talha (acacia seyal), and cultured plant cell gums including those of the plants of the genera: acacia, actinidia, aptenia, carbobrotus, chickorium, cucumis, glycine, hibiscus, hordeum, letuca, lycopersicon, malus, medicago, mesembryanthemum, oryza, panicum, phalaris, phleum, poliathus, polycarbophil, sida, solanum, trifolium, trigonella, Afzelia africana seed gum, Treculia africana gum, detarium gum, cassia gum, carob gum, Prosopis africana gum, Colocassia esulenta gum, Hakea gibbosa gum, khaya gum, scleroglucan, zea, mixtures of any of the foregoing, and the like.

In some aspects, the compositions comprising an inhibitor of mTOR are formed into nanoparticles and subsequently encapsulated or coated. In some embodiments, the encapsulant or coating may be an enteric coating. In some embodiments, the encapsulated rapamycin nanoparticles provide rapamycin nanoparticles within a protective polymer matrix for oral administration of rapamycin. The result is not only more durable and stable, but is also more bioavailable and efficacious for treatment and prevention of genetically-predisposed disorders and age-related disorders, especially in the fields of oncology and neurology in humans and other animals.

The encapsulated rapamycin nanoparticles provide an embodiment of the present invention in the form of an improved form of encapsulated rapamycin that is more durable, stable and bioavailable. In some embodiments, the encapsulated rapamycin provides the rapamycin nanoparticles within a controlled release matrix, forming the encapsulated rapamycin nanoparticle in a single drug delivery structure for oral administration of rapamycin. This encapsulated rapamycin nanoparticle may also be referred to as an enteric-coated rapamycin nanoparticle. In addition, many of the embodiments also include a stabilizing compound (for our purposes, a “stabilizer”) within the controlled release matrix either to improve compatibility of the rapamycin with the controlled release matrix, to stabilize the crystalline morphology of the rapamycin, or to help further prevent degradation of the rapamycin, particularly when the encapsulated rapamycin nanoparticle is exposed to air, atmospheric moisture, or room temperature or warmer conditions. Particular embodiments incorporate the stabilizers within each rapamycin nanoparticle, although certain aspects of the invention may be embodied with stabilizers on the surface of the encapsulated rapamycin nanoparticles or otherwise dispersed in the controlled release matrix. To different levels depending on the particular approach used for producing the nanoparticles, with or without other additives, the result is more efficacious for treatment and prevention of genetically-predisposed disorders and age-related disorders, especially in the fields of oncology and neurology in humans and other animals.

Rapid anti-solvent precipitation, or controlled precipitation, is one method of preparing the rapamycin nanoparticles as it provides for minimal manipulation of the rapamycin and exquisite control over nanoparticle size and distribution, and the crystallinity of the rapamycin. Several controlled precipitation methods are known in the art, including rapid solvent exchange and rapid expansion of supercritical solutions, both of which can be implemented in batch or continuous modes, are scalable, and suitable for handling pharmaceutical compounds.

Rapamycin nanoparticles prepared by controlled precipitation methods can be stabilized against irreversible aggregation, Ostwald ripening, and/or reduced dispersibility, by control of colloid chemistry, particle surface chemistry and particle morphology. For example, nanoparticles prepared by antisolvent solidification can be stabilized by ionic and non-ionic surfactants that adsorb to nanoparticle surfaces and promote particle colloid stability through either charge repulsion or steric hindrance, respectively. Moreover, stabilizers can affect nanoparticle crystallinity, which may be used to promote different biodistribution and bioavailability in certain indications.

Rapamycin nanoparticles can consist of molecular rapamycin bound by suitable methods to other nanoparticles. Suitable methods of attaching rapamycin to a nanoparticle carrier or substrate may include physical adsorption through hydrogen van der Waals forces or chemisorption through covalent or ionic bonding. Nanoparticle substrates may be either natural or synthetic, and modified to promote specific interactions with rapamycin. Natural nanoparticles include albumin and other proteins, and DNA. Synthetic nanoparticles include organic and inorganic particulates, micelles, liposomes, dendrimers, hyperbranched polymers, and other compounds.

The rapamycin nanoparticles can be processed by any suitable method, such as by milling, high-pressure atomization, or rapid anti-solvent precipitation. Milling is suitable provided care is taken to minimize both rapamycin degradation and particle agglomeration. Rapamycin degradation can be reduced with the aid of cooling or cryogenic processes. Agglomeration due to the increased surface area and concomitant adhesive forces can be reduced by the use of dispersants during the milling process.

In some embodiments, the rapamycin nanoparticles are sized between about 1 nanometer and about 1 micron. In some embodiments, the rapamycin nanoparticles are less than 1 micron diameter. Such smaller particles provide better control of final particle size, improved stability within the particles, and the ability to tune bioavailability by controlling the crystallinity and composition of the rapamycin nanoparticles.

Manufacturing approaches for the encapsulated rapamycin nanoparticle drug delivery structure embodiments of the present invention include creating a solution of the controlled release matrix, with the rapamycin nanoparticles dispersed therein, in appropriate proportion and producing a heterogeneous mixture. The solvent for such mixtures can be a suitable volatile solvent for the controlled release matrix. In some embodiments, the solvent is either a poor solvent or non-solvent for the rapamycin nanoparticles so that when the rapamycin nanoparticles are dispersed into the controlled release matrix solution they remain as discrete nanoparticles. The resulting dispersion of rapamycin nanoparticles in the controlled release matrix solution can then be reduced to a dry particulate powder by a suitable process, thereby resulting in microparticles of a heterogeneous nature comprised of rapamycin nanoparticles randomly distributed in the controlled release matrix. The particulate powder may also be tailored by a suitable process to achieve a desired particle size for subsequent preparation, which may be from about 20 to about 70 microns in diameter.

The rapamycin nanoparticles are microencapsulated with the controlled release matrix using a suitable particle-forming process to form the encapsulated rapamycin nanoparticle. An example of a particle-forming process is spinning disk atomization and drying. For a detailed discussion of the apparatus and method concerning the aforementioned spin disk coating-process, this application incorporates by references US Patent Applications 2011/221337 and 2011/220430, respectively. Alternatively, for example, the encapsulated rapamycin nanoparticles can be prepared by spray drying.

In some embodiments, not all of the rapamycin nanoparticles will be encapsulated within the controlled release matrix. Instead the rapamycin nanoparticles may be enmeshed with the controlled release matrix, with some of the rapamycin nanoparticles wholly contained within the controlled release matrix while another other rapamycin nanoparticles apparent on the surface of the drug delivery structure, constructed in appearance similar to a chocolate chip cookie.

In some embodiments, and depending on the size of the rapamycin nanoparticles, the encapsulated rapamycin nanoparticles are between 10 and 50 microns in diameter, although diameters as large as 75 microns may be suitable.

The controlled release matrix of the encapsulated rapamycin nanoparticles can be selected to provide desired release characteristics of the encapsulated rapamycin nanoparticles. For example, the matrix may be pH sensitive to provide either gastric release or enteric release of the rapamycin. Enteric release of the rapamycin may achieve improved absorption and bioavailability of the rapamycin. Many materials suitable for enteric release are known in the art, including fatty acids, waxes, natural and synthetic polymers, shellac, and other materials. Polymers are a one enteric coating and may include copolymers of methacrylic acid and methyl methacrylate, copolymers of methyl acrylate and methacrylic acid, sodium alginate, polyvinyl acetate phthalate, and various succinate or phthalate derivatives of cellulose and hydroxpropyl methyl cellulose. Synthetic polymers, such as copolymers of methacrylic acid and either methyl acrylate or methyl methacrlate, are good enteric release polymers due the ability to tune the dissolution pH range of these synthetic polymers by adjusting their comonomer compositions. Examples of such pH sensitive polymers are EUDRAGIT® polymers (Evonik Industries, Essen, Germany). Specifically, EUDRAGIT® S-100, a methyl methacrylate and methacrylic acid copolymer with comonomer ratio of 2:1, respectively, has a dissolution pH of about 7.0, thereby making is suitable for enteric release of rapamycin.

The encapsulated rapamycin nanoparticles may be delivered in various physical entities including a pill, tablet, or capsule. The encapsulated rapamycin nanoparticles may be pressed or formed into a pellet-like shape and further encapsulated with a coating, for instance, an enteric coating. In another embodiment, the encapsulated rapamycin nanoparticles may be loaded into a capsule, also further enterically coated.

Various performance enhancing additives can be added to the encapsulated rapamycin nanoparticles. For example, additives that function as free radical scavengers or stabilizers can be added to improve oxidative and storage stability of the encapsulated rapamycin nanoparticles. In some embodiments, free radical scavengers are chosen from the group that consists of glycerol, propylene glycol, and other lower alcohols. Additives alternatively incorporate antioxidants, such as α-tocopherol (vitamin E), citric acid, EDTA, α-lipoic acid, or the like.

Methacrylic acid copolymers with methyl acrylate or methyl methacrylate are moderate oxygen barriers. Furthermore, these polymers will exhibit an equilibrium moisture content. Oxygen transport due to residual solvent, moisture or other causes, can lead to degradation of the encapsulated rapamycin nanoparticles. Oxygen barrier materials can be added to the encapsulated rapamycin nanoparticles formulation to improve oxygen barrier properties. Oxygen barrier polymers compatible with the polymers are polyvinyl alcohol (PVA) and gelatin.

### F. Microparticle and Nanoparticle Rapamycin

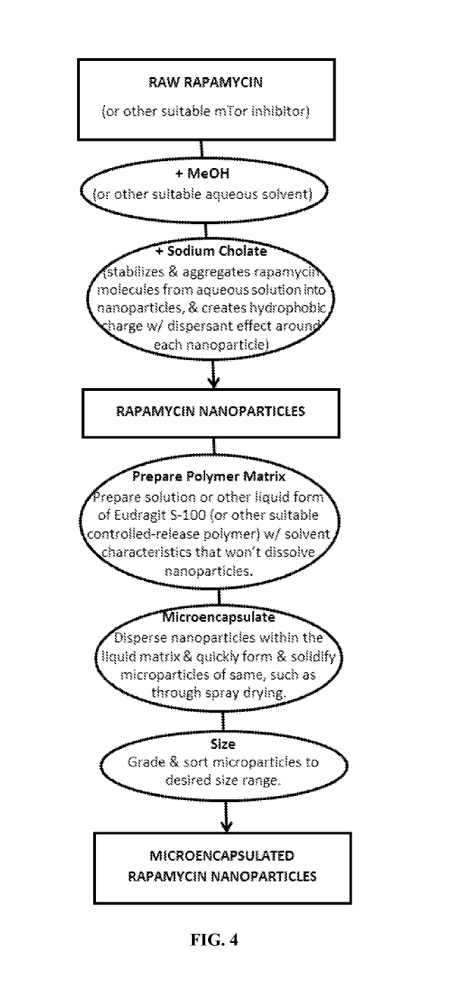

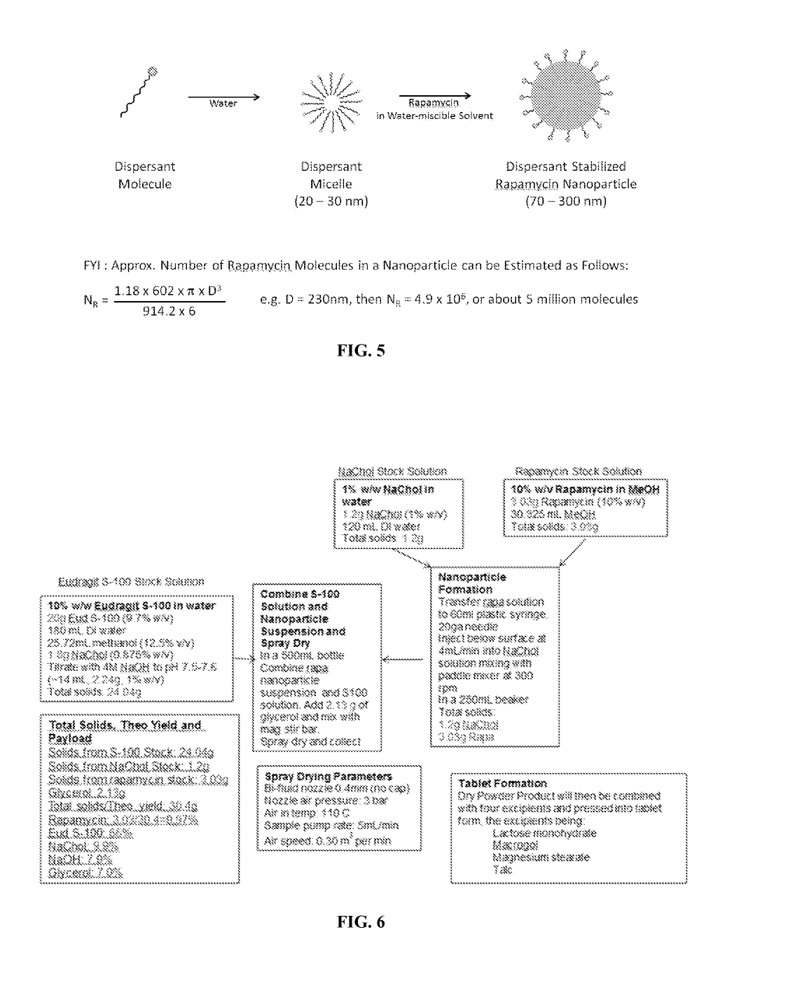

In some embodiments, rapamycin nanoparticle inclusions comprise discrete nanoparticles of rapamycin heterogeneously dispersed in a controlled release matrix. As illustrated in FIGS. 4-6, the rapamycin nanoparticles are prepared by a suitable method and may contain additives to promote nanoparticle stability, modify rapamycin crystallinity, or promote compatibility of the rapamycin nanoparticles with the controlled release matrix. The controlled release matrix is formulated to promote release of rapamycin to specific parts of the body, such as the intestine, to enhance oxidative and storage stability of the encapsulated rapamycin nanoparticles, and to maintain the discrete, heterogeneously distributed nature of the rapamycin nanoparticles.

Referring to FIG. 3, rapamycin nanoparticles are prepared by anti-solvent precipitation or solidification, also sometimes referred to as controlled precipitation or solidification. Antisolvent solidification is one approach as it provides exquisite control of particle size and distribution, particle morphology, and rapamycin crystallinity. For example, it is possible to prepare nanoparticles with narrow particle size distribution that are amorphous, crystalline, or combinations thereof. Such properties may exhibit additional benefits, by further controlling the biodistribution and bioavailability of rapamycin in specific indications.

Referring now to FIG. 4, rapamycin is dissolved in a suitable water-miscible solvent and then rapidly injected into rapidly stirred water containing an appropriate aqueous soluble dispersant. Water-miscible solvents for rapamycin include methanol, ethanol, isopropyl alcohol, acetone, dimethylsulfoxide, dimethylacetamide, n-methylpyrolidone, tetrahydrofuran, and other solvents. Low boiling point, high vapor pressure water-miscible solvents facilitate their removal during subsequent microparticle formation. Examplary water-miscible solvents are methanol, acetone, and isopropyl alcohol. In some embodiments, the water-miscible solvent is methanol. Some aqueous soluble dispersants include ionic surfactants such as sodium dodecyl sulfate and sodium cholate, non-ionic surfactants such as Pluronics, Poloxomers, Tweens, and polymers, such as polyvinyl alcohol and polyvinylpyrolidone. Examplary aqueous-soluble dispersants are sodium cholate, Pluronic F-68, and Pluronic F-127. In some embodiments, the aqueous-soluble dispersant is sodium cholate, which provides surprisingly beneficial properties. Not only is sodium cholate a surfactant and a dispersant, it serves to cause aggregation of rapamycin particles from the aqueous solution. Moreover, while sodium cholate tends to be a polar molecule as well as an amphoteric surfactant, it surrounds each nanoparticle with a hydrophobic charge when it is enmeshed in the Eudragit matrix. Then, when the nanoparticle is released from the Eudragit matrix within the animal subject's enteric passages where conditions are basic, the same properties cause the nanoparticle to be more readily received and absorbed through the intestinal walls.

Referring to FIG. 5 now, rapamycin is dissolved in the water-miscible solvent at a concentration of about 0.01% w/v to about 10.0% w/v preferably about 0.1% w/v to about 1.0% w/v. The aqueous-soluble dispersant is dissolved in water at a concentration above its critical micelle concentration, or CMC, typically at about 1 to about 10 times the CMC. The rapamycin solution is injected into the aqueous-soluble dispersant solution with agitation at a volumetric ratio of about 1:10 to about 1:1, preferably about 1:5 to about 1:1.

The controlled release matrix is prepared from a water-soluble polymer, which may be a copolymer of methacrylic acid with either methyl acrylate or methyl methacrylate, such as those marketed under the trade name of EUDRAGIT® and having pH-dependent dissolution properties. The controlled release matrix may be comprised of EUDRAGIT® S-100, although other water-soluble enteric controlled release would be suitable. Water-soluble controlled release matrices are selected so as either not to compromise the integrity of rapamcyin nanoparticles or to provide a medium in which rapamycin nanoparticles may be prepared by the controlled precipitation methodology described previously.

In preparing the water-soluble polymer it is helpful to maintain conditions that do not compromise the integrity of the rapamycin nanoparticles. Firstly, since the rapamycin nanoparticles are susceptible solubilization by certain co-solvents, it is important to maintain a suitable quantity of certain co-solvents to achieve controlled release matrix solubility while not deleteriously affecting the morphology of the rapamycin nanoparticles. Secondly, rapamycin nanoparticles will be susceptible to chemical degradation by high pH; therefore, it is important to modulate the controlled release matrix solution pH so that rapamycin is not chemically altered. It is helpful the controlled release matrix solution pH be maintained below about pH 8. Lastly, it is helpful to achieve near to complete solubilization of the controlled release matrix in solution so that microencapsulation of the rapamycin nanoparticles by the controlled release matrix in subsequent processing steps may proceed with high efficiency. When using the EUDRAGIT® S-100 as the controlled release matrix, it is helpful to achieve a controlled release matrix solution by using a combination of co-solvents and solution pH modulation. In certain embodiments, the co-solvents are about 40% or less by volume. Similarly, in certain embodiments, the pH of the controlled release matrix solution is about 8 or less, such that the EUDRAGIT® S-100 is not completely neutralized and may be only about 80% or less neutralized. These conditions achieve nearly complete to complete solubilization of the EUDRAGIT® S-100 in a medium that is mostly aqueous and that maintains the integrity of the rapamycin nanoparticles, therefore leading to their microencapsulation by the controlled-release matrix in subsequent processing steps.

The rapamycin nanoparticles prepared by the controlled precipitation method are added to the aqueous solution of the controlled released matrix, resulting in a nanoparticle dispersion in the solubilized controlled release matrix. Alternatively, the rapamycin solubilized in a suitable co-solvent can be dispersed into the aqueous solution of controlled release matrix leading to controlled precipitation of rapamycin particles, thereby leading to a rapamycin nanoparticle dispersion in fewer processing steps, but of appropriate composition to permit subsequent microencapsulation processing.

As an alternative embodiment, the encapsulated rapamycin nanoparticles are created using pre-existing nanoparticle substrates, such as albumin, to create, in the case of albumin, “albumin-rapamycin nanoparticles.” Within this general class of alternatives, certain approaches for creating the albumin-rapamycin nanoparticles involve encapsulating rapamycin within albumin nanoparticles or preferentially associating rapamycin with albumin nanoparticles through physical or chemical adsorption. The albumin nanoparticles themselves may be formed from human serum albumin, a plasma protein derived from human serum.

More particularly, this embodiment may involve use of a therapeutic peptide or protein that is covalently or physically bound to albumin, to enhance its stability and half-life. With the albumin stabilized, the rapamycin is mixed with the stabilized albumin in an aqueous solvent and passed under high pressure to form rapamycin-albumin nanoparticles in the size range of 100-200 nm (comparable to the size of small liposomes).

Certain embodiments also address degradation risks and other limits imposed by the related art by preparing encapsulated rapamycin nanoparticles as a heterogeneous mixture of rapamycin nanoparticles in a polymer matrix. Distributed nanoparticles are morphologically different than homogeneous rapamycin; and are less susceptible to degradation because of the bulk nature of the nanoparticles compared to the smaller size of molecular rapamycin.

### G. Methods of Using Rapamycin Compositions

“Treatment” and “treating” refer to administration or application of a therapeutic agent to a subject or performance of a procedure or modality on a subject for the purpose of obtaining a therapeutic benefit for a disease or health-related condition. For example, the rapamycin compositions of the present invention may be administered to a subject for the purpose of preventing cerebrovascular function dysfunction in a patient who has been identified as an ApoE4 carrier.

The terms “therapeutic benefit,” “therapeutically effective,” or “effective amount” refer to the promotion or enhancement of the well-being of a subject. This includes, but is not limited to, a reduction in the frequency or severity of the signs or symptoms of a disease.

“Prevention” and “preventing” are used according to their ordinary and plain meaning. In the context of a particular disease or health-related condition, those terms refer to administration or application of an agent, drug, or remedy to a subject or performance of a procedure or modality on a subject for the purpose of preventing or delaying the onset of a disease or health-related condition. For example, one embodiment includes administering the rapamycin compositions of the present invention to a subject at risk for developing an endocrine tumor or endocrine cancer for the purpose of preventing cerebrovascular function dysfunction in a patient who has been identified as an ApoE4 carrier.

Rapamycin compositions, as disclosed herein, may be used to treat any disease or condition for which an inhibitor of mTOR is contemplated as effective for treating or preventing the disease or condition. For example, methods of using rapamycin compositions to preventing cerebrovascular dysfunction in a patient who has been identified as an ApoE4 carrier are disclosed. Identification of a patient as an ApoE4 carrier may be determined by genetic analysis. The treatment or prevention of the disease may be instituted before or after any related surgical or medical intervention. Dosing regimens may include multiple doses per day, one dose per day, or regular doses one or more days apart.

Other uses of rapamycin compositions as disclosed herein are also contemplated. For example, U.S. Pat. No. 5,100,899 discloses inhibition of transplant rejection by rapamycin; U.S. Pat. No. 3,993,749 discloses rapamycin antifungal properties; U.S. Pat. No. 4,885,171 discloses antitumor activity of rapamycin against lymphatic leukemia, colon and mammary cancers, melanocarcinoma and ependymoblastoma; U.S. Pat. No. 5,206,018 discloses rapamycin treatment of malignant mammary and skin carcinomas, and central nervous system neoplasms; U.S. Pat. No. 4,401,653 discloses the use of rapamycin in combination with other agents in the treatment of tumors; U.S. Pat. No. 5,078,999 discloses a method of treating systemic lupus erythematosus with rapamycin; U.S. Pat. No. 5,080,899 discloses a method of treating pulmonary inflammation with rapamycin that is useful in the symptomatic relief of diseases in which pulmonary inflammation is a component, i.e., asthma, chronic obstructive pulmonary disease, emphysema, bronchitis, and acute respiratory distress syndrome; U.S. Pat. No. 6,670,355 discloses the use of rapamycin in treating cardiovascular, cerebral vascular, or peripheral vascular disease; U.S. Pat. No. 5,561,138 discloses the use of rapamycin in treating immune related anemia; U.S. Pat. No. 5,288,711 discloses a method of preventing or treating hyperproliferative vascular disease including intimal smooth muscle cell hyperplasia, restenosis, and vascular occlusion with rapamycin; and U.S. Pat. No. 5,321,009 discloses the use of rapamycin in treating insulin dependent diabetes mellitus.

### H. Pharmaceutical Preparations

Certain methods and compositions set forth herein are directed to administration of an effective amount of a composition comprising the rapamycin compositions of the present invention.

1. Compositions

A “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (Remington's, 1990). Except insofar as any conventional carrier is incompatible with the active ingredient, its use in the therapeutic or pharmaceutical compositions is contemplated. The compositions used in the present invention may comprise different types of carriers depending on whether it is to be administered in solid, liquid or aerosol form, and whether it needs to be sterile for such routes of administration as injection.

The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions, and these are discussed in greater detail below. For human administration, preparations should meet sterility, pyrogenicity, general safety and purity standards as required by FDA Office of Biologics standards.

The formulation of the composition may vary depending upon the route of administration. For parenteral administration in an aqueous solution, for example, the solution should be suitably buffered if necessary and the liquid diluent first rendered isotonic with sufficient saline or glucose. In this connection, sterile aqueous media that can be employed will be known to those of skill in the art in light of the present disclosure.

In addition to the compounds formulated for parenteral administration, such as intravenous or intramuscular injection, other pharmaceutically acceptable forms include, e.g., tablets or other solids for oral administration; liposomal and nanoparticle formulations; enteric coating formulations; time release capsules; formulations for administration via an implantable drug delivery device, and any other form. One may also use nasal solutions or sprays, aerosols or inhalants in the present invention.

The capsules may be, for example, hard shell capsules or soft-shell capsules. The capsules may optionally include one or more additional components that provide for sustained release.

In certain embodiments, pharmaceutical composition includes at least about 0.1% by weight of the active compound. In other embodiments, the pharmaceutical composition includes about 2% to about 75% of the weight of the composition, or between about 25% to about 60% by weight of the composition, for example, and any range derivable therein.

The compositions may comprise various antioxidants to retard oxidation of one or more components. Additionally, the prevention of the action of microorganisms can be accomplished by preservatives such as various antibacterial and antifungal agents, including but not limited to parabens (e.g., methylparabens, propylparabens), chlorobutanol, phenol, sorbic acid, thimerosal or combinations thereof. The composition should be stable under the conditions of manufacture and storage, and preserved against the contaminating action of microorganisms, such as bacteria and fungi.

In certain embodiments, an oral composition may comprise one or more binders, excipients, disintegration agents, lubricants, flavoring agents, and combinations thereof. When the dosage unit form is a capsule, it may contain, in addition to materials of the above type, carriers such as a liquid carrier. Various other materials may be present as coatings or to otherwise modify the physical form of the dosage unit. For instance, tablets, pills, or capsules may be coated with shellac, sugar or both.

In particular embodiments, prolonged absorption can be brought about by the use in the compositions of agents delaying absorption, such as, for example, aluminum monostearate, gelatin, or combinations thereof.

2. Routes of Administration

Upon formulation, solutions will be administered in a manner compatible with the dosage formulation and in such amount as is therapeutically effective.

The composition can be administered to the subject using any method known to those of ordinary skill in the art. For example, a pharmaceutically effective amount of the composition may be administered intravenously, intrathecally, intracerebrally, intracranially into the cortex, thalamus, hypothalamus, hippocampus, basal ganglia, substantia nigra or the region of the substantia nigra, intradermally, intraarterially, intraperitoneally, intralesionally, intratracheally, intranasally, topically, intramuscularly, intraperitoneally, subcutaneously, orally, topically, locally, inhalation (e.g., aerosol inhalation), injection, infusion, continuous infusion, localized perfusion bathing target cells directly, via a catheter, via a lavage, in creams, in lipid compositions (e.g., liposomes), or by other method or any combination of the forgoing as would be known to one of ordinary skill in the art (Remington's, 1990).

In particular embodiments, the composition is administered to a subject using a drug delivery device. Any drug delivery device is contemplated for use in delivering an effective amount of the inhibitor of mTORC 1.

3. Dosage

A pharmaceutically effective amount of an inhibitor of mTORC1 is determined based on the intended goal. The quantity to be administered, both according to number of treatments and dose, depends on the subject to be treated, the state of the subject, the protection desired, and the route of administration. Precise amounts of the therapeutic agent also depend on the judgment of the practitioner and are peculiar to each individual.

The amount of rapamycin or rapamycin analog or derivative to be administered will depend upon the disease to be treated, the length of duration desired and the bioavailability profile of the implant, and the site of administration. Generally, the effective amount will be within the discretion and wisdom of the patient's physician. Guidelines for administration include dose ranges of from about 0.01 mg to about 500 mg of rapamycin or rapamycin analog.

For example, a dose of the inhibitor of mTORC1 may be about 0.0001 milligrams to about 1.0 milligrams, or about 0.001 milligrams to about 0.1 milligrams, or about 0.1 milligrams to about 1.0 milligrams, or even about 10 milligrams per dose or so. Multiple doses can also be administered. In some embodiments, a dose is at least about 0.0001 milligrams. In further embodiments, a dose is at least about 0.001 milligrams. In still further embodiments, a dose is at least 0.01 milligrams. In still further embodiments, a dose is at least about 0.1 milligrams. In more particular embodiments, a dose may be at least 1.0 milligrams. In even more particular embodiments, a dose may be at least 10 milligrams. In further embodiments, a dose is at least 100 milligrams or higher.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

The dose can be repeated as needed as determined by those of ordinary skill in the art. Thus, in some embodiments of the methods set forth herein, a single dose is contemplated. In other embodiments, two or more doses are contemplated. In some embodiments, the two or more doses are the same dosage. In some embodiments, the two or more doses are different dosages. Where more than one dose is administered to a subject, the time interval between doses can be any time interval as determined by those of ordinary skill in the art. For example, the time interval between doses may be about 1 hour to about 2 hours, about 2 hours to about 6 hours, about 6 hours to about 10 hours, about 10 hours to about 24 hours, about 1 day to about 2 days, about 1 week to about 2 weeks, or longer, or any time interval derivable within any of these recited ranges. In specific embodiments, the composition may be administered daily, weekly, monthly, annually, or any range therein.

Doses for encapsulated rapamycin (eRapa) and for encapsulated rapamycin nanoparticles maybe different. According to certain embodiments, doses are contemplated in a range of more than 50 micrograms and up to (or even exceeding) 200 micrograms per kilogram for daily administration, or the equivalent for other frequencies of administration. Although dosing may vary based on particular needs and preferred treatment protocols according to physician preference, maximum tolerable daily bioavailable dosings (trough levels) for a 28-day duration are about 200 micrograms of rapamycin (or equivalent) per subject kilogram, for both human and canine subjects, although those of ordinary skill would understand that greater dose amount ranges would be tolerable and suitable when administered less often than once per day, and lesser ranges would be tolerable when administered more often than once per day.

In certain embodiments, it may be desirable to provide a continuous supply of a pharmaceutical composition to the patient. This could be accomplished by catheterization, followed by continuous administration of the therapeutic agent. The administration could be intra-operative or post-operative.

4. Secondary and Combination Treatments

Certain embodiments provide for the administration or application of one or more secondary or additional forms of therapies. The type of therapy is dependent upon the type of disease that is being treated or prevented. The secondary form of therapy may be administration of one or more secondary pharmacological agents that can be applied in preventing cerebrovascular function dysfunction in a patient who has been identified as an ApoE4 carrier or a disease, disorder, or condition associated with cerebrovascular function dysfunction in a patient who has been identified as an ApoE4 carrier.

If the secondary or additional therapy is a pharmacological agent, it may be administered prior to, concurrently, or following administration of the inhibitor of mTORC1.

The interval between administration of the inhibitor of mTORC1 and the secondary or additional therapy may be any interval as determined by those of ordinary skill in the art. For example, the inhibitor of mTORC1 and the secondary or additional therapy may be administered simultaneously, or the interval between treatments may be minutes to weeks. In embodiments where the agents are separately administered, one would generally ensure that a significant period of time did not expire between the time of each delivery, such that each therapeutic agent would still be able to exert an advantageously combined effect on the subject. For example, the interval between therapeutic agents may be about 12 h to about 24 h of each other or within about 6 hours to about 12 h of each other. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations. In some embodiments, the timing of administration of a secondary therapeutic agent is determined based on the response of the subject to the inhibitor of mTORC 1.

### I. Kits

Kits are also contemplated as being used in certain aspects of the present invention. For instance, a rapamycin composition of the present invention can be included in a kit. A kit can include a container. Containers can include a bottle, a metal tube, a laminate tube, a plastic tube, a dispenser, a pressurized container, a barrier container, a package, a compartment, or other types of containers such as injection or blow-molded plastic containers into which the hydrogels are retained. The kit can include indicia on its surface. The indicia, for example, can be a word, a phrase, an abbreviation, a picture, or a symbol.

Further, the rapamycin compositions of the present invention may also be sterile, and the kits containing such compositions can be used to preserve the sterility. The compositions may be sterilized via an aseptic manufacturing process or sterilized after packaging by methods known in the art.

### EXAMPLES

The following examples are included to demonstrate certain non-limiting aspects of the invention. It should be appreciated by those of skill in the art that the techniques disclosed in the examples that follow represent techniques discovered by the inventors to function well in the practice of the invention. However, those of skill in the art should, in light of the present disclosure, appreciate that many changes can be made in the specific embodiments that are disclosed and still obtain a like or similar result without departing from the spirit and scope of the invention.

### Example 1

Functional neuroimaging was employed to evaluate changes in cerebral blood flow (CBF) in mouse models that are associated with a decline in memory and increased depression. In particular, mouse models of atherosclerosis and Alzheimer's disease (AD), as well as aging rats were studied.

Magnetic resonance imaging (MRI) has shown that cerebral blood flow (CBF) declines with age in humans, and this age-related decline is accelerated in AD. It has also been observed that individuals with the ApoeE4 allele, who are at greater risk for AD, also show decreased CBF before any decline in cognition is observed. Therefore, cerebrovascular dysfunction, which can be assessed by CBF, may lead to blood-brain-barrier defects that initiate the generation of pro-inflammatory cytokines and the cascade of events leading to plaque formation along with tangles and loss of memory.

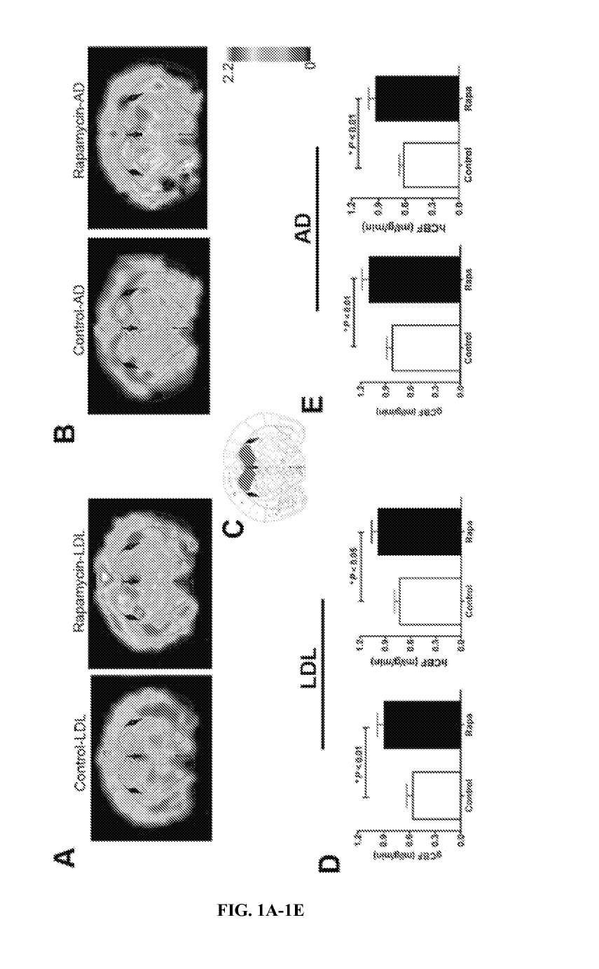

Experiments using high-field arterial spin label (ASL) MRI show that AD-transgenic mice that were fed encapsulated rapamycin (14 ppm) (a dosage that improved memory) exhibit significantly increased CBF (FIGS. 1B and 1E). ASL was performed on a horizontal 7T/30 cm magnet and a 40G/cm BGA12S gradient insert (Bruker, Billerica, Mass.). A small circular surface coil (ID=1.1 cm) was placed on top of the head and a circular labeling coil (ID=0.8 cm), built into the cradle, was placed at the heart position for CASL. The two coils were positioned parallel to each other, separated by 2 cm from center to center, and were actively decoupled. Paired images were acquired in an interleaved fashion with field of view (FOV)=12.8×12.8 mm², matrix=128×128, slice thickness=1 mm, 9 slices, labeling duration=2100 ms, TR=3000 ms, and TE=20 ms. ASL image analysis employed codes written in Matlab and STIMULATE software (University of Minnesota) to obtain CBF.

To assess whether rapamycin could improve CBF in a different mouse model, LDL-receptor knockout mice (LDLr−/−) were fed a high-fat diet, which provides a mouse model of atherosclerosis. Feeding rapamycin to these mice improved performance of the LDLr−/−mice fed a high-fat diet in the Morris Water Maze and increased CBF in whole brain.

The Morris Water Maze experiments were performed as follows. All animals showed no deficiencies in swimming abilities, directional swimming or climbing onto a cued platform during pre-training and had no sensorimotor deficits as determined with a battery of neurobehavioral tasks performed prior to testing. All groups were assessed for swimming ability 2 days before testing. The procedure described by Morris et al. 1984 was followed as described in Spilman et al. 2010; Pierce et al. 2012; and Galvan et al. 2006. Experimenters were blind with respect to genotype and treatment. Briefly, mice were given a series of 6 trials, 1 hour apart in a light-colored tank filled with opaque water whitened by the addition of non-toxic paint at a temperature of 24.0±1.0° C. In the visible portion of the protocol, mice were trained to find a 12×12-cm submerged platform (1 cm below water surface) marked with a colored pole that served as a landmark placed in different quadrants of the pool. The animals were released at different locations in each 60′ trial. If mice did not find the platform in 60 seconds, they were gently guided to it. After remaining on the platform for 20 seconds, the animals were removed and placed in a dry cage under a warm heating lamp. Twenty minutes later, each animal was given a second trial using a different release position. This process was repeated a total of 6 times for each mouse, with each trial ˜20 minutes apart. In the non-cued part of the protocol, the water tank was surrounded by opaque dark panels with geometric designs at approximately 30 cm from the edge of the pool, to serve as distal cues. The animals were trained to find the platform with 6 swims/day for 5 days following the same procedure described above. At the end of training, a 45-second probe trial was administered in which the platform was removed from the pool. The number of times that each animal crossed the previous platform location was determined as a measure of platform location retention. During the course of testing, animals were monitored daily, and their weights were recorded weekly. Performance in all tasks was recorded by a computer-based video tracking system (Water2020, HVS Image, U.K). Animals that spent more than 70% of trial time in thigmotactic swim were removed from the study. Data were analyzed offline by using HVS Image and processed with Microsoft Excel before statistical analyses.

Rapamycin improved performance of the LDLr−/−mice fed a high-fat diet in the Morris Water Maze and increased CBF in whole brain. These results indicate that chronic rapamycin treatment can ameliorate spatial memory deficits, and that this is associated with increased CBF in mice modeling atherosclerosis. The data shown in FIG. 1 demonstrate that rapamycin significantly improves global and hippocampal CBF in LDLr−/−mice and in mice modeling AD.

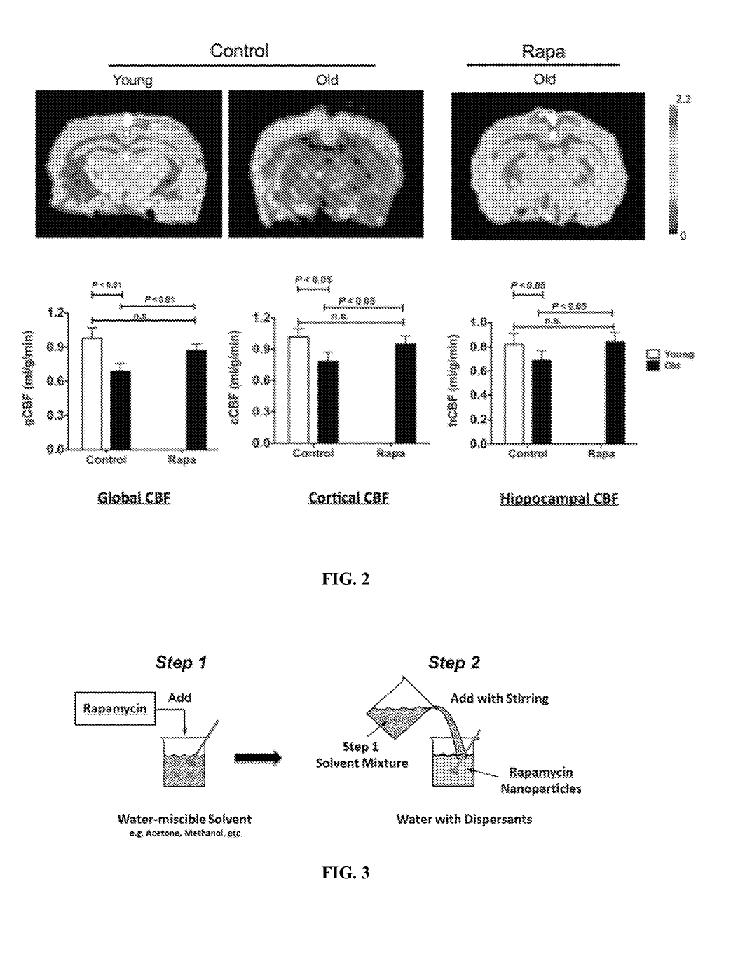

Rapamycin effects on CBF were also assessed in aged rats. As shown in FIG. 2, 32 month-old rats who were fed rapamcyin for 16 weeks exhibited improved global CBF, cortical CBF, and hippocampal CBF as compared to aged rats who did not receive rapamycin treatment. These improvements in CBF were associated with improved spatial learning in rapamycin-treated aged rats as determined using the Morris Water Maze task.

The ability of rapamycin to restore vascular function in Alzheimer's mice, LDL-receptor knockout mice on a high-fat diet, and older rats indicates that rapamycin can also prevent cerebral vascular dysfunction and/or restore cerebrovascular function in ApoE4 carriers. This is because CBF is observed as an early phenotype in ApoE4 carriers that is thought to initiate the disorders that ApoE4 carriers develop. By preventing cerebrovascular dysfunction, rapamycin would prevent or reduce the risk of disorders that ApoE4 carriers are prone to developing, including non-AD dementia, age-related cognitive dysfunction, stroke, depression, and cerebral palsy. In addition, the observed effects of rapamycin on vascular function further indicate that rapamycin would be effective in treating ApoE4 carriers exposed to traumatic brain injury (TBI) with and without hemorrhagic brain trauma.

### Example 2

Development of methods to produce rapamycin nanoparticles. Rapid solvent exchange was used to examine the formation of rapamycin nanoparticles. Three water-miscible solvents and three water-soluble surfactants were selected to study their respective effects on the formation and morphology of rapamycin nanoparticles. The water-miscible solvents were isopropyl alcohol (Solvent 1), acetone (Solvent 2), and methanol (Solvent 3). The water-soluble surfactants were Pluronic F-68 (Dispersant 1, a non-ionic PEO-PPO-PEO block copolymer), Pluronic F-127 (Dispersant 2, a non-ionic PEO-PPO-PEO block copolymer), and sodium cholate (Dispersant 3, an anionic surfactant). Rapamycin was dissolved in each of the water-miscible solvents at a concentration of 0.25% w/v. The water-soluble surfactants were dissolved in deionized water at concentrations of 0.5% w/v, 0.5% w/v, and 1.0% w/v, respectively, for each of the dispersants. Each experimental combination (e.g. NP-1 to NP-9 in following table) consisted of 5 mL of rapamycin solution and 25 mL of surfactant solution, resulting in a dilution factor of 1:5 solvent:water. 25 mL of surfactant solution was transferred to a 50 mL beaker and stirred with the aid of magnetic mircostirbar. Rapamycin solution was rapidly injected at 500 uL increments with the aid of a micropipette with the pipette tip placed below the surface of the rapidly stirred surfactant solution. The visual appearance of the resulting nanoparticles and their colloidal stability after 24-hours were qualitatively assessed. The following table summarizes the qualities of the rapamycin nanoparticle dispersions. Qualitatively, rapamycin nanoparticle dispersions having a colorless to blue, opalescent appearance will have particle sizes on the order of less than about 300 nm as evidenced by their interaction with the ultraviolet wavelengths of visible light. Whereas, dispersions having a more white appearance will have particle sizes larger than about 300 nm due to their interaction with the broader spectrum of visible light. Rapamycin nanoparticle formulations NP-7 and NP-9 were selected as methods of nanoparticle preparation.

| | Dispersant 1 | Dispersant 2 | Dispersant 3 |
|---|---|---|---|
| Solvent 1 | NP-1: White, | NP-2: Blue, | NP-3: Clear, |
| | settled, | opalescent, settled, | aggregated, |
| | resdispersible | redispersible | redispersible |
| Solvent 2 | NP-4: Blue, | NP-5: White, | NP-6: Blue, |
| | opalescent, some | settled, | opalescent, |
| | settling | redispersible | settled, |
| | | | redispersible |
| Solvent 3 | NP-7: Blue, | NP-8: Blue to white, | NP-9: Blue, |
| | opalescent, stable | settled, redispersible | opalescent, stable |

### Example 3

Preparation of a high concentration rapamycin nanoparticle dispersion. The water-miscible solvent and water-soluble dispersant of NP-9 from Example 2 was used to prepare rapamycin nanoparticles. 656 mg of rapamycin were dissolved in 6.56 mL of Solvent 3 to yield a 1.0% w/v solution. This volume of rapamycin solution was injected into 26.25 mL of 1.0% w/v Dispersant 1 in deionized water. The resulting rapamycin nanoparticle dispersion had a final rapamycin content of 2.4% w/w. The particle size of the dispersion was determined by dynamic light scattering to be 230 nm±30 nm with a single peak.

### Example 4

Preparation of a water-soluble enteric coating. 3.5 g of EUDRAGIT® S-100 were added to 70 mL of deionized water with light stirring, resulting in a white dispersion. 1.4 g of sodium hydroxide were added to the dispersion with continued stirring. The resulting dispersion gradually turned clear and colorless indicating an aqueous solution of S-100. The estimated concentration of sodium hydroxide was 0.5N.

### Example 5

Preparation of a feedstock containing rapamycin nanoparticles and a water-soluble enteric coating. Rapamycin nanoparticles were prepared as described in Example 3 and then slowly added to an aqueous solution of EUDRAGIT® S-100 prepared as in Example 4. The ratio of rapamycin to S-100 was 1:9, or 10% wt. rapamycin payload. The resulting dispersion was allowed to stir for several minutes to observe stability. After one hour, the dispersion had transformed to a clear yellow, indicating destruction of the rapamycin nanoparticles and a change in the rapamycin. Addition of a small amount of acetic acid to reduce the solution pH to below neutral resulted in a clear, colorless solution.

### Example 6

Preparation of water-soluble enteric coating with a water-miscible co-solvent. 3.5 g of EUDRAGIT® 5-100 were added to 30/70 v/v methanol/deionized water, resulting in a white dispersion. The dispersion was stirred continuously until a clear solution was formed.

### Example 7

Preparation of a feedstock containing rapamycin nanoparticles and a water-soluble enteric coating. Rapamycin nanoparticles were prepared as described in Example 3 and then slowly added to an aqueous solution of EUDRAGIT® S-100 prepared as in Example 6. The ratio of rapamycin to S-100 was 1:9, or 10% wt. rapamycin payload. The white dispersion was allowed to stir for several minutes after which the dispersion was transformed into a clear solution indicating the rapamycin nanoparticles had been destroyed.

### Example 8

Preparation of a partially-neutralized, water-soluble enteric coating with a water-miscible co-solvent. 3.5 g of EUDRAGIT® S-100 were added to 10/90 v/v methanol/deionized water, resulting in a white dispersion. The dispersion was titrated to clarity with 2.000 mL of 4.8M sodium hydroxide. The estimated neutralization of the S-100 was 78%.

### Example 9

Preparation of a feedstock containing rapamycin nanoparticles and a water-soluble enteric coating. Rapamycin nanoparticles were prepared as described in Example 2 then slowly added to an aqueous solution of EUDRAGIT® S-100 as prepared in Example 7. The ratio of rapamycin to S-100 was 1:9, or 10% wt. rapamycin payload. The resulting white dispersion remained stable for several hours as indicated by no change in color or change in optical clarity. The final pH was 7.5. The particle size of the final dispersion was determined by dynamic light scattering to be 756 nm±52 nm with a single peak and indicating possible clustering of the rapamycin nanoparticles in the resulting feedstock.

### Example 10

Preparation of a feedstock containing rapamycin nanoparticles and a water-soluble enteric coating. The rapamycin solution used in Example 3 was injected with stirring into the aqueous solution of EUDRAGIT® S-100 prepared in Example 8. The ratio of rapamycin to S-100 was 1:9, or 10% wt. rapamycin payload. A blue, opalescent colloid was formed and it remained stable for several hours as indicated by no change in color or change in optical clarity. The final pH was 7.5. The particle size of the final dispersion was determined by dynamic light scattering to be 305 nm±60 nm with a single peak.

### Example 11

Spray drying of feedstock containing rapamycin nanoparticles and a water-soluble enteric coating. The feedstocks prepared in Examples 9 and 10 were spray dried and analyzed for rapamycin content. Particles prepared from Example 9 had a rapamycin content of 9.5% wt. (87% rapamycin yield). Particles prepared from Example 10 had a rapamycin content of 7.9% wt. (80% rapamycin yield).

### Example 12

Storage stability of enteric-coated encapsulated rapamycin nanoparticles. Microparticles prepared by spray drying in Example 11 were stored under controlled conditions at room temperature and 50% relative humidity. Samples were analyzed weekly for rapamycin content. All samples maintained at least 95% of their original rapamycin content at all time points for at least three weeks.

### Example 13

Preparation of nanoparticles in Eudragit S-100. Referring to FIG. 6, a rapamycin solution was prepared by combining rapamycin with methanol in a 10% w/v ratio as 3.03 g rapamycin and 30.25 ml methanol. A 1% w/w sodium cholate solution was prepared by combining 1.2 g sodium cholate with 120 ml deionized water. Nanoparticle formation was achieved by transferring the rapamycin solution with a 60 ml plastic syringe equipped with a 20 ga needle, injecting the rapamycin solution below the surface of the sodium cholate solution in a 250 ml beaker. Mixing was accomplished with a paddle mixer operating at 300 rpm yielding a rapamycin nanoparticle suspension. A 10% w/w Eudragit S-100 solution was prepared by combining 20 g Eudragit S-100 in a 9.7% w/v mixture with 180 ml deionized water, 25.72 ml methanol in a 12.5% v/v mixture, and 1.8 g sodium cholate in a 0.875% w/v mixture. This 10% w/w Eudragit S-100 solution was titrated with 4M sodium hydroxide to achieve a pH of between about 7.5 and about 7.6. Encapsulated rapamycin particles were then fabricated by combining the Eudragit S-100 solution with the rapamycin nanoparticle suspension. The Eudragit S-100 solution and the rapamycin nanoparticle suspension were combined in a 500 ml bottle, adding 2.13 g of glycerol and mixing with a magnetic stir bar. The combined Eudragit S-100 solution and rapamycin nanoparticle suspension were then spray dried and collected. The spray drying parameters included a 0.4 mm nozzle, nozzle air pressure of 3 bar, input air temperature of 110° C., a sample pump rate of 5 ml/min and an air speed of 0.30 m3/min.

### REFERENCES

The following references, to the extent that they provide exemplary procedural or other details supplementary to those set forth herein, are specifically incorporated herein by reference.
Fryer, et al., J Neurosci. 23(21): 7889-96, 2003.
Galvan et al. Proc Natl Acad Sci USA. 103(18): 7130-5, 2006.
Graziani, et al., Nat Prod Rep. 26:602-9, 2009.
Gregory et al., Angwandte Chemie. 116(19): 2605-7, 2004.
Gregory et al., Org Biomol Chem. 4: 3565-8, 2006.
Iadecola, Nat Rev Neurosci. 5(5): 347-60, 2004.
Morris et al. Neurosci Methods. 11(1): 47-60, 1984.
Pierce et al. J Neurochem, 124(6):880-93, 2012.
Schwecke et al., PNAS USA. 92(17): 7839-43, 1995.
Spilman et al. PLoS One. 5(4): e9979, 2010.
U.S. Pat. No. 4,316,885
U.S. Pat. No. 5,023,262
U.S. Pat. No. 5,023,263
U.S. Pat. No. 5,023,264
U.S. Pat. No. 5,100,883
U.S. Pat. No. 5,102,876
U.S. Pat. No. 5,118,677
U.S. Pat. No. 5,118,678
U.S. Pat. No. 5,120,725
U.S. Pat. No. 5,120,726
U.S. Pat. No. 5,120,727
U.S. Pat. No. 5,120,842
U.S. Pat. No. 5,130,307
U.S. Pat. No. 5,138,051
U.S. Pat. No. 5,151,413
U.S. Pat. No. 5,162,333
U.S. Pat. No. 5,164,399
U.S. Pat. No. 5,169,851
U.S. Pat. No. 5,177,203
U.S. Pat. No. 5,194,447
U.S. Pat. No. 5,202,332
U.S. Pat. No. 5,221,670
U.S. Pat. No. 5,221,740
U.S. Pat. No. 5,233,036
U.S. Pat. No. 5,260,299
U.S. Pat. No. 5,260,300
U.S. Pat. No. 5,262,423
U.S. Pat. No. 5,262,424
U.S. Pat. No. 5,302,584
U.S. Pat. No. 5,310,903
U.S. Pat. No. 5,344,833
U.S. Pat. No. 5,346,893
U.S. Pat. No. 5,358,944
U.S. Pat. No. 5,362,718
U.S. Pat. No. 5,373,014
U.S. Pat. No. 5,378,696
U.S. Pat. No. 5,378,836
U.S. Pat. No. 5,385,908
U.S. Pat. No. 5,385,909
U.S. Pat. No. 5,385,910
U.S. Pat. No. 5,389,639
U.S. Pat. No. 5,391,730
U.S. Pat. No. 5,411,967
U.S. Pat. No. 5,434,260
U.S. Pat. No. 5,446,048
U.S. Pat. No. 5,463,048
U.S. Pat. No. 5,480,988
U.S. Pat. No. 5,480,989
U.S. Pat. No. 5,484,790
U.S. Pat. No. 5,484,791
U.S. Pat. No. 5,486,522
U.S. Pat. No. 5,486,523
U.S. Pat. No. 5,486,524
U.S. Pat. No. 5,488,054
U.S. Pat. No. 5,489,595
U.S. Pat. No. 5,489,680
U.S. Pat. No. 5,491,231
U.S. Pat. No. 5,504,091
U.S. Pat. No. 5,504,204
U.S. Pat. No. 5,504,291
U.S. Pat. No. 5,508,285
U.S. Pat. No. 5,508,286
U.S. Pat. No. 5,508,290
U.S. Pat. No. 5,508,399
U.S. Pat. No. 5,516,780
U.S. Pat. No. 5,519,031
U.S. Pat. No. 5,521,194
U.S. Pat. No. 5,525,610
U.S. Pat. No. 5,530,007
U.S. Pat. No. 5,530,121
U.S. Pat. No. 5,532,355
U.S. Pat. No. 5,541,191
U.S. Pat. No. 5,541,192
U.S. Pat. No. 5,550,133
U.S. Pat. No. 5,559,112
U.S. Pat. No. 5,559,119
U.S. Pat. No. 5,559,120
U.S. Pat. No. 5,559,122
U.S. Pat. No. 5,563,145
U.S. Pat. No. 5,567,709
U.S. Pat. No. 5,637,590
U.S. Pat. No. 5,665,772
U.S. Pat. No. 5,780,462
U.S. Pat. No. 5,912,253
U.S. Pat. No. 5,922,730
U.S. Pat. No. 5,955,457
U.S. Pat. No. 5,985,890
U.S. Pat. No. 6,004,973
U.S. Pat. No. 6,015,809
U.S. Pat. No. 6,399,625
U.S. Pat. No. 6,440,990
U.S. Pat. No. 6,677,357
U.S. Pat. No. 6,680,330

1. A method for preventing cerebrovascular dysfunction in a patient comprising administering an effective amount of a composition comprising rapamycin or an analog thereof to a patient who has been identified as an ApoE4 carrier.

2. The method of claim 1, wherein preventing the cerebrovascular function dysfunction prevents Alzheimer's Disease (AD), non-AD dementia, age-related cognitive dysfunction, stroke, depression, or cerebral palsy.

3. A method of treating traumatic brain injury (TBI) in a patient comprising administering an effective amount of a composition comprising rapamycin or an analog thereof to a patient who has been identified as an ApoE4 carrier.

4. The method of any of claim 1, wherein the rapamycin or analog thereof is encased in a coating comprising cellulose acetate succinate, hydroxy propyl methyl cellulose phthalate co-polymer, or a polymethacrylate-based copolymer selected from the group consisting of methyl acrylate-methacrylic acid copolymer, and a methyl methacrylate-methacrylic acid copolymer.

5. The method of claim 4, wherein the coating comprises Poly(methacylic acid-co-ethyl acrylate) in a 1:1 ratio, Poly(methacrylic acid-co-ethyl acrylate) in a 1:1 ratio, Poly(methacylic acid-co-methyl methacrylate) in a 1:1 ratio, Poly(methacylic acid-co-methyl methacrylate) in a 1:2 ratio, Poly(methyl acrylate-co-methyl methacrylate-co-methacrylic acid) in a 7:3:1 ratio, Poly(ethyl acrylate-co-methyl methacrylate-co-trimethylammonioethyl methacrylate chloride) in a 1:2:0.2 ratio, Poly(ethyl acrylate-co-methyl methacrylate-co-trimethylammonioethyl methacrylate chloride) in a 1:2:0.1 ratio, or Poly(butyl methacylate-co-(2-dimethylaminoethyl) methacrylate-co-methyl methacrylate) in a 1:2:1 ratio, a naturally-derived polymer, or a synthetic polymer, or any combination thereof.

6. The method of claim 5, wherein the naturally-derived polymer is selected from the group consisting of alginates and their various derivatives, chitosans and their various derivatives, carrageenans and their various analogues, celluloses, gums, gelatins, pectins, gellans, polyethyleneglycols (PEGs) and polyethyleneoxides (PEOs), acrylic acid homo- and copolymers with acrylates and methacrylates, homopolymers of acrylates and methacrylates, polyvinyl alcohol (PVOH), and polyvinyl pyrrolidone (PVP).

7. (canceled)

8. The method of any of claim 1, wherein the composition comprises rapamycin or an analog thereof at a concentration of 0.001 mg to 30 mg total per dose.

9. The method of any of claim 1, wherein the composition comprising rapamycin or an analog of rapamycin comprises 0.001% to 60% by weight of rapamycin or an analog of rapamycin.

10. The method of any of claim 1, wherein the average blood level of rapamycin in the subject is greater than 0.5 ng/mL blood after administration of the composition.

11. The method of any of claim 1, wherein the composition is administered orally, intravenously, enterically, or intranasally.

12. The method of any of claim 1, wherein the rapamycin or analog of rapamycin is administered in two or more doses.

13. The method of claim 12, wherein the interval of time between administration of doses comprising rapamycin or an analog of rapamycin is 0.5 to 30 days.

14. The method of claim 1, wherein the subject is further administered a composition comprising a second active agent, wherein the second active agent comprises eNOS, a cholinesterase inhibitor, an anti-glutamate, an anti-hypertensive agent, an anti-platelet agent, an antihyperlipidemic agent, an anti-anxiety agent, an anti-depressant agent, an antipsychotic agent, an anti-seizure agent, an anti-Parkinson agent, an anti-spasmodic agent, an anti-tremor agent, a muscle relaxant agent, or a medication that alleviates or treats low blood pressure, cardiac arrhythmia, or diabetes; or a biological agent that includes an antibody or antibodies to neurofibrillary tangles or cerebral plaques.

15. (canceled)

16. The method of claim 14, wherein the anti-cholinesterase therapeutic is tacrine, donepezil, rivastigmine, galantamine, or a humanized antibody, protein, or RNA sequence.

17. The method of claim 14, wherein the anti-glutamate therapeutic is memantine, or a humanized antibody, protein or RNA sequence.

18. The method of claim 14, wherein the biologic agent to neurofibrillary tangles or cerebral plaques is a polyclonal antibody or humanized monoclonal antibody, protein or RNA sequence.

19. The method of claim 14, wherein the composition comprising rapamycin or an analog of rapamycin is administered at the same time as the composition comprising the second active agent.

20. The method of claim 14, wherein the composition comprising rapamycin or an analog of rapamycin is administered before or after the composition comprising the second active agent is administered.

21. The method of claim 20, wherein the interval of time between administration of the composition comprising rapamycin or an analog of rapamycin and the composition comprising the second active agent is 1 to 30 days.

22. The method of claim 1, wherein the composition comprising rapamycin or an analog of rapamycin is comprised in a food or food additive.
